(19) United States
(12) Patent Application Publication
(10) Pub. No.: US 2024/0000820 A1
(43) Pub. Date: Jan. 4, 2024

(54) A METHOD OF TREATING FOOT ROT

(71) Applicant: Intervet Inc., Madison, NJ (US)

(72) Inventors: Luis Fernando Vettorato, Paulinia (BR); Mauricio Marques De Morais, Batatais (BR); Rafael Marin Chiummo, Mainz (DE); Heitor De Oliveira Arriero Amaral, São Paulo (BR)

(73) Assignee: Intervet Inc., Madison, NJ (US)

(21) Appl. No.: 18/254,401

(22) PCT Filed: Dec. 2, 2021
(86) PCT No.: PCT/EP2021/083899

§ 371 Date: May 25, 2023

(30) Foreign Application Priority Data
Dec. 3, 2020 (EP) 20211553.1

(51) Int. Cl. A61K 31/706; A61K 9/00; A61K 9/08; A61K 47/12; A61K 47/10; A61P 31/04

(52) U.S. Cl. CPC A61K 31/706; A61K 9/0019; A61K 9/08; A61K 47/12; A61K 47/10; A61P 31/04

(57) ABSTRACT

The subject invention is the novel use of an injectable pharmaceutical composition comprising tildipirosin for the treatment and prophylaxis of foot rot. The use of a single subcutaneous dose of tildipirosin has been demonstrated to effectively treat naturally occurring foot rot.

### BACKGROUND

Foot rot disorders in cattle are necrotic diseases that affect the connective tissue of the interdigital region and are caused by bacteria, mainly Fusobacterium necrophorum. Foot rot disorders are also commonly found in sheep, goats and swine. These disorders rot away the foot of the animal, specifically, the area between the two toes of the affected animal. They are extremely painful and contagious. Foot infections impact animal productivity and fertility and causes great economic losses, especially those animals raised in intensive feed lot systems.

Tildipirosin is a semi-synthetic macrolide antibiotic and is the active ingredient in Zuprevo®. The IUPAC name for tildipirosin is (4R,5S,6S,7R,9R,11E,13E,15R,16R)-6-[(2R,3R,4S,5S,6R)-4-(dimethylamino)-3,5-dihydroxy-6-methyloxan-2-yl]oxy-16-ethyl-4-hydroxy-5,9,13-trimethyl-7-(2-piperidin-1-ylethyl)-15-(piperidin-1-ylmethyl)-1-oxacyclohexadeca-11,13-diene-2,10-dione and the structure is

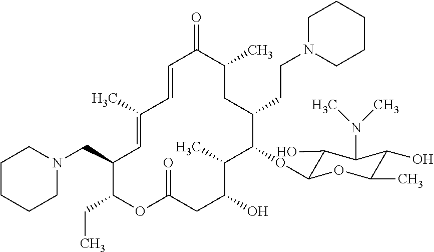

Zuprevo® is a ready-to-use sterile injectable solution containing 18% tildipirosin, a semi-synthetic macrolide antibiotic, for treatment and prophylaxis of Bovine Respiratory Disease. Each mL of Zuprevo® contains 180 mg of tildipirosin as the free base, 82.5 mg citric acid monohydrate and 400 mg propylene glycol, and water qs with citric acid monohydrate added to adjust pH. This pharmaceutical composition is used for the treatment and prevention of bovine respiratory disease (BRD) associated with Mannheimia haemolytica, Pasteurella multocida and Histophilus somni sensitive to tildipirosin. See Freedom of Information, NADA 141-334, May 14, 2012.

Zuprevo® 4% tildipirosin injectable solution is available for the treatment of swine respiratory disease (SRD) associated with Actinobacillus pleuropneumoniae, Pasteurella multocida, Bordetella bronchiseptica and Haemophilus parasuis. This solution comprises tildipirosin as a free base in a concentration of 40 mg/mL, propylene glycol, citric acid monohydrate and water. See EPAR Summary for the Public, EMA/166222/2011, updated February 2015.

WO2008/012343 discloses processes to make tildipirosin. WO 2009/013351 discloses solvated and non-solvated crystalline forms of tildipirosin. U.S. Pat. No. 6,514,946 discloses the structure of tildipirosin and its use to treat various livestock and poultry diseases.

Recent studies have shown that tildipirosin was rapidly absorbed and slowly eliminated after a single subcutaneous administration in healthy lambs. It was proposed that tildipirosin could be used for the treatment and prevention of respiratory bacterial infections in sheep (see Abu-Basha, E A, et al., Pharmacokinetics and bioavailability of tildipirosin following intravenous and subcutaneous administration in sheep. J vet Pharmacol Therap. 2020; 00: 1-7. htts://doi.or/101111/jvp.1901).

None of these references discloses the use of tildipirosin to treat or prevent foot rot diseases.

### SUMMARY OF THE INVENTION

An embodiment of the invention is an injectable pharmaceutical composition comprising tildipirosin, a salt or solvate thereof and a pharmaceutically acceptable carrier for use in the treatment and/or prevention of foot rot disease in an animal.

### DESCRIPTION OF THE FIGURES

FIG. 1 presents the lameness data from the tildipirosin group and the control group.

FIG. 2 presents the lesion data from the tildipirosin group and the control group.

### DETAILED DESCRIPTION

The subject invention is the novel use of an injectable pharmaceutical composition comprising tildipirosin for the treatment and prevention of foot rot. The use of a single subcutaneous dose of tildipirosin injectable solution at the dose of 4 mg of tildipirosin per kg of bodyweight (BW) has been demonstrated to effectively treat naturally occurring foot rot.

The treatment of foot rot is useful on intensively reared beef cattle such as those in feedlot confinement operations. The treatment is intended to reduce hoof lesions and lameness, reduce infections and prevent new infections from bacteria such as F. necrophorum. The invention can have significant impact on farm productivity and animal welfare. The invention is convenient to the farm management since it consists of a single treatment which reduces the need of restraining animals. Furthermore, tildipirosin is also used to treat and prevent Bovine Respiratory Disease (BRD), so that a single treatment can help control two of the most important diseases on intensively reared beef cattle.

The invention is also useful in the treatment of foot rot in swine with same advantages of convenience of administration and treatment or prevention of two disease with a single treatment as noted above for cattle.

An embodiment of the invention is an injectable pharmaceutical composition comprising tildipirosin, a salt or solvate thereof and a pharmaceutically acceptable carrier for use in the treatment and/or prevention of foot rot disease in an animal.

In an embodiment of the invention, the pharmaceutically acceptable carrier comprises one or more excipients.

In an embodiment of the invention, the injectable pharmaceutical composition is a solution or a suspension.

In an embodiment of the invention, the injectable pharmaceutical composition is a solution.

In an embodiment of the invention, the excipients are one or more solvents.

In an embodiment of the invention, the solvents are propylene glycol, water or mixtures thereof.

In an embodiment of the invention, the tildipirosin is the form of the free base.

In an embodiment of the invention, the pharmaceutical composition further comprises an acid, preferably citric acid.

In an embodiment of the invention, the dose of tildipirosin is from about 1 mg/kg to about 10 mg/kg of body weight of the animal or from about 3 mg/kg to about 7 mg/kg or preferably about 4 mg/kg.

In an embodiment of the invention, the animal is a sheep, a goat, a bovid or a swine, preferably a bovid.

In an alternative embodiment of the invention, the animal is a swine.

In an alternative embodiment of the invention, the animal is a sheep.

In an alternative embodiment of the invention, the animal is a goat.

In an embodiment of the invention, the concentration of tildipirosin is from about 10% w/v to about 25% w/v or from about 15% w/v to about 20% w/v or preferably 18% w/v.

In an embodiment of the invention, the concentration of tildipirosin is from about 0.1% w/v to about 9.9% w/v or from about 2% w/v to about 6% w/v or preferably 4% w/v.

An embodiment of the invention is an injectable pharmaceutical composition for use in the treatment and/or prevention of foot rot disease in an animal, wherein each mL of the composition comprises
a) 180 mg of tildipirosin as the free base;
b) 82.5 mg citric acid monohydrate;
c) 400 mg propylene glycol; and
d) water qs.

In an embodiment of the invention, the animal is a bovid.

In an embodiment of the invention, the injectable pharmaceutical composition is a suspension.

In an embodiment of the invention, the injectable pharmaceutical composition comprises a salt or a solvate of tildipirosin.

An alternative embodiment of the invention is a method of treating foot rot in an animal comprising administering a pharmaceutical composition comprising an effective amount of tildipirosin to the animal.

In another embodiment, the pharmaceutical composition is administered intravenously.

In another embodiment, the method comprises a single administration of the pharmaceutical composition.

In another embodiment, the effective amount of tildipirosin is from about 1 mg/kg to about 10 mg/kg of body weight of the animal.

In another embodiment, wherein the effective amount of tildipirosin is from about 3 mg/kg to about 7 mg/kg, preferably about 4 mg/kg.

In another embodiment, the animal is a sheep, a goat, a bovid or a swine.

In another embodiment, the pharmaceutical composition further comprises one or more excipients.

In another embodiment, the tildipirosin is in the form of the freebase.

In another embodiment, the pharmaceutical composition comprises a solvent selected from propylene glycol, water or mixtures thereof.

In another embodiment, the pharmaceutical composition comprises an acid.

In another embodiment, the acid is citric acid.

In another embodiment, the use is a single injection.

### Example 1

This study demonstrated the efficacy of tildipirosin injectable solution (Zuprevo®) for use in bovine animals, given subcutaneously at one single dose of 4 mg of the active ingredient/kg body weight (BW) against naturally occurring foot rot.

Thirty animals were enrolled to the Control Group and another thirty animals were enrolled in the Tildipirosin treated Group. The tildipirosin-treated group received a single dose, subcutaneously, while the control group was treated with a saline sterile solution. Both groups (treated and control) were housed in the same paddocks under typical Brazilian feedlot conditions.

Sixty animals were included in the trial in two different phases, according to the number of available animals that fulfill the inclusion criteria. The study was masked. That is the study personnel making post-treatment clinical assessments including lameness did not know the treatment assignments. The animals were randomized and evenly distributed across the treatment groups according to 15 blocks of 2 animals each in both inclusion dates (there were 2 inclusion dates, for a total of 60 animals).

The effectiveness of tildipirosin for the treatment of foot rot was evaluated by comparing the proportion of treatment success/improvement in the tildipirosin-treated group to the saline-treated control group on Day 7.

There were no abnormal health observations before or after treatment. One adverse event was observed with one animal of the tildipirosin-treated group that had a broken toenail on Day 7. According to the investigator, the adverse event was not related to the tildipirosin treatment, and this animal was removed from the study.

The lameness and the lesion scoring are described in Tables 1 and 2 below.

TABLE 1
Lameness Scoring guide

| Score | Clinical | |
|---|---|---|
| value | Description | Assessment Criteria |
| 1 | Normal | Animal stands and walks with a level-back posture |
| | | and clinically normal gait. |
| 2 | Mildly | Animal stands with a level-back posture but |
| | lame | develops an arched-back posture during walking. |
| | | The gait remains clinically normal. |
| 3 | Moderately | Animal has an arched-back posture that is evident |
| | lame | during standing and walking. Animal has a short- |
| | | strided gait in 1 or more limbs. |
| 4 | lame | Animal always has an arched-back posture and |
| | | gait is one deliberate step at a time. Animal favors |
| | | one or more limbs or feet. |
| 5 | Severely | Animal additionally demonstrates an inability |
| | lame | or extreme reluctance to bear weight on one or |
| | | more limbs or feet. |

TABLE 2
Lesion Scoring guide

| Score | |
|---|---|
| value | Lesion Description |
| 1 | No abnormality observed. |
| 2 | Slight to moderate swelling present. Evidence of healing lesion |
| | with granulation tissue present throughout the majority of the |
| | lesion. |
| 3 | Severe swelling with no necrotic lesion; or slight swelling with |
| | small interdigital necrotic lesion covering up to 10% of the |
| | interdigital space. |
| 4 | Small to medium size necrotic lesion covering >10% up to 50% |
| | of the interdigital space with moderate to severe swelling. |
| 5 | Large to very large interdigital necrotic lesion covering >50% |
| | up to100% of the interdigital space with moderate to severe |
| | swelling. |

The animals' lesion and lameness score were assessed by the investigator on Days −1 and 0 of the study. To qualify for the effectiveness portion of the study, animals were diagnosed with foot rot by meeting the following criteria:
Lameness Score 3 in at least one foot for 2 consecutive days.
Lesion Score 2 in at least one foot for 2 consecutive days.
Clinically healthy (excepting lameness)
Body weight (348-499 kg body weight)
Age (20-24 months)

On Day 0, before treatment, bacteriological samples were collected from the infected hooves of the enrolled cattle. Lesions and lameness were also assessed on Days 2, 4 and 7. Treatment success, improvement, or failure was determined on Day 7 and was based on lameness and lesion score compared to Day 0. On Day 7, another bacteriological sample was collected from all animals.

Bacteriological samples were collected on a sterile scraper and on a sterile swab from the border of healthy and necrotic tissue (around the hoof lesion) of the infected hooves of the enrolled cattle. The tip of the scraper, containing discharge from the lesions, was fractured off into 15 ml tubes containing 7 ml of a semi solid transport media formulated exclusively to anaerobic bacteria and the swab was discharged in a semi solid transport. The tubes were kept under refrigeration (4 to 8° C.) until they arrive into the laboratory. At the laboratory, the samples grew in culture media appropriated to F. necrophorum and Dichelobacter nodosus isolation. Isolates were identified using confirmatory biochemical tests and polymerase chain reaction (PCR) specific to F. necrophorum and D. nodosus species.

The tildipirosin-treated group demonstrated decreased symptoms scores for both lameness and lesion. In the treated group, the animals had an average lameness score of 3.1 on Day 0. The lameness score of this group decreased to 1.3 on Day 7. For the lesion score, the treated group animals had an average score of 2.3 on Day 0 which decreased to a score of 1.9 on Day 7. In contrast, the control group's average lameness score of 3.1 on Day 0 decreased to only 2.4 on Day 7. Their average lesion score of 2.3 on Day 0 increased slightly to a score of 2.4 on Day 7.

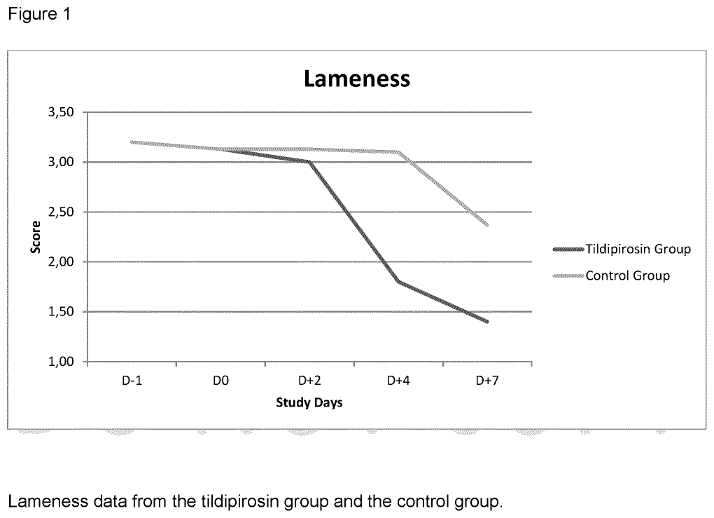

FIG. 1 presents the lameness score data. Both the treated group and the control group had the same mean score (3.1). After treatment each group behaved differently. The score of the tildipirosin treated animals decreased to 1.3, while the score of the control group stayed in 2.4. Clearly, the tildipirosin treatment produced a better score evaluation in the tildipirosin treated animals as shown in FIG. 1.

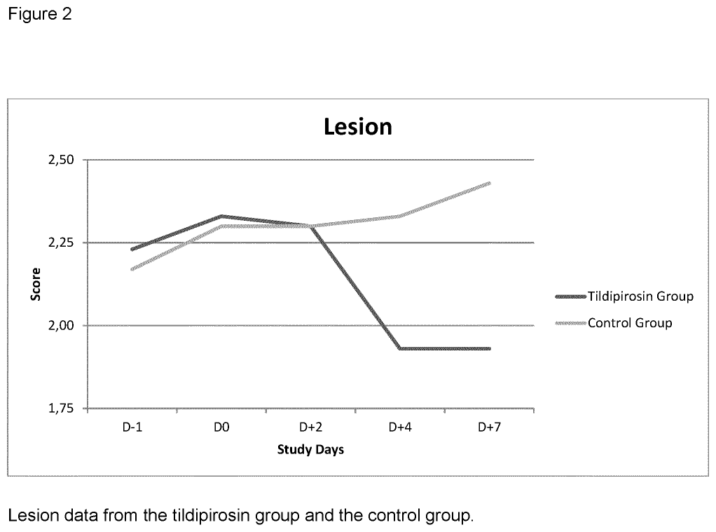

FIG. 2 displays the lesion score data. Before treatment, the lesion score was similar for both groups: tildipirosin-treated group (2.3) and control group (2.2). However, there is a visible difference in the behavior of each group following evaluations at Day 7. The control group increased the lesion score (2.4), in other words it got worse, and the tildipirosin treated animals decreased the lesion score (1.9), getting closer to a clinical resolution.

The bacterial evaluation was not conclusive, although more animals treated with tildipirosin that were positive to PCR identification of Fusobacterium necrophorum before treatment administration became negative after treatment and also less tildipirosin treated animals that were previously negative became positive, both in comparison to the control group.

1. A method of treating or preventing foot rot disease in a bovid animal comprising administering to the bovid animal an injectable pharmaceutical composition comprising tildipirosin, a salt or solvate thereof and a pharmaceutically acceptable carrier.

2. The method of claim 1, wherein the pharmaceutically acceptable carrier comprises one or more excipients.

3. The method of claim 1, wherein the injectable pharmaceutical composition is a solution or a suspension.

4. The method of claim 3, wherein the injectable pharmaceutical composition is a solution.

5. The method of claim 4, wherein the excipients are one or more solvents.

6. The method of claim 5, wherein the solvents are propylene glycol, water or mixtures thereof.

7. The method of claim 1, wherein the tildipirosin is the form of the free base.

8. The method of claim 7, wherein the pharmaceutical composition further comprises an acid.

9. The method of claim 8, wherein the acid is citric acid.

10. The method of claim 1, wherein the dose of the tildipirosin is from about 1 mg/kg to about 10 mg/kg of body weight of the animal or from about 3 mg/kg to about 7 mg/kg or preferably about 4 mg/kg.

11. The method of claim 4, wherein the concentration of the tildipirosin is from about 10% w/v to about 25% w/v, from about 15% w/v to about 20% w/v, preferably 18% w/v.

12. The method of claim 1, wherein each mL of the composition comprises
a) 180 mg of tildipirosin as the free base;
b) 82.5 mg citric acid monohydrate;
c) 400 mg propylene glycol; and
d) water qs.

13. The method of claim 3, wherein the injectable pharmaceutical composition is a suspension.

14. The method of claim 13, comprising a salt or a solvate of tildipirosin.

15. The method of claim 1, wherein the injectable pharmaceutical composition is administered as a single injection.
